(19) United States
(12) Patent Application Publication
(10) Pub. No.: US 2024/0218400 A1
(43) Pub. Date: Jul. 4, 2024

(54) CORN OIL YIELD ENHANCEMENT THROUGH USE OF EMULSIFIERS AND EMULSIONS BY-PRODUCTS OF FERMENTATION

(71) Applicant: POET RESEARCH, INC., Sioux Falls, SD (US)

(72) Inventors: Cory J. Sarks, Sioux Falls, SD (US); Alex T. Mccurdy, Sioux Falls, SD (US); Andrew Manning, Sioux Falls, SD (US); Steven T. Bly, Sioux Falls, SD (US)

(21) Appl. No.: 18/555,290

(22) PCT Filed: Apr. 15, 2022
(86) PCT No.: PCT/US2022/025046

§ 371 Date: Oct. 13, 2023

Related U.S. Application Data
(60) Provisional application No. 63/175,639, filed on Apr. 16, 2021.

(51) Int. Cl. C12P 7/06; C12N 9/10; C12P 7/6436

(52) U.S. Cl. CPC C12P 7/06; C12N 9/1051; C12P 7/6436; C12Y 204/01021

(57) ABSTRACT

Methods of enhancing corn oil recovery from a bio-refinery using recycled by-products of the process including emulsifiers in the form of emulsions, defatted emulsions, precipitates thereof, and/or dried products thereof.

### CROSS-REFERENCE TO RELATED APPLICATION

This application claims the benefit of U.S. Provisional Application No. 63/175,639, filed on Apr. 16, 2021, which is hereby incorporated by reference in its entirety.

### TECHNICAL FIELD

Provided herein are methods, compositions, and uses of by-products obtained from bio-refinery operations for production of a target chemical, such as ethanol, by fermenting a feedstock, such as corn, for corn oil yield enhancement

### BACKGROUND

Ethanol can be produced from grain-based feedstocks (corn, sorghum, milo, barley, wheat, soybeans, etc.), from sugar (sugar cane, sugar beets, etc.), or from biomass (lignocellulosic feed stocks, switchgrass, corn cobs, stover, wood, or other plant material). In a conventional ethanol plant, corn is used as a feedstock and ethanol is produced from starch contained within the corn. Typically, corn kernels are cleaned and milled to prepare starch-containing material for processing. The corn kernels can also be fractionated to separate the starch-containing material (endosperm) from other matter (such as fiber and germ). The starch-containing material is commonly slurried with water and liquefied to facilitate saccharification, where the starch is converted into sugar (glucose), and fermentation, where the sugar is converted by an ethanologen (yeast) into ethanol. The fermentation product is often referred to as beer, which includes a liquid component including ethanol, water, and soluble components, and a solids component including unfermented particulate matter (among other things). The fermentation product is sent to a distillation system where the fermentation product is distilled and dehydrated into ethanol. The residual matter (whole stillage) includes water, soluble components, oil, and unfermented solids (the solids component of the beer with substantially all ethanol removed, which can be dried into dried distillers grains (DDG) and sold, for example, as an animal feed product). Other co-products (syrup and oil contained in the syrup), can also be recovered from the whole stillage. Water removed from the fermentation product in distillation can be treated for re-use at the plant.

In the case of ethanol production from corn, the syrup co-product contains an amount of corn oil, which can be separated and sold as distiller's corn oil (DCO). Alternatively, corn oil can be separated prior to fermentation, from the beer, from the whole stillage, from the thin stillage, from the wet cake or any other corn oil containing process stream. Bio-refineries may separate DCO from process streams using, for instance, centrifuges to produce the corn oil product. Various processes for recovering the corn oil from the fermentation products noted above are currently known in the art. Such processes, however, can be expensive, inefficient, or produce low quality oil. Conventional processes for recovering oil from a fermentation product can sacrifice oil quality or oil quantity such that the oil contains a high level of free fatty acids or oil may be lost in various other processing streams. The presence of a high level of free fatty acids can hamper the production of end products. Current processes to recovery oil as a by-product of ethanol fermentation may be limited in the amount of high quality corn oil that can be recovered therefrom.

### SUMMARY

In one approach or embodiment, a method for enhancing the corn oil yield in a bio-refinery fermentation process is described herein. The method includes obtaining an alcohol and by-products including vegetable oil from a grain feedstock through a processing that includes saccharification, fermentation, distillation, and separation to produce the alcohol and the vegetable oil. Next, an emulsifier in the form of emulsions, defatted emulsions, emulsion precipitates, defatted emulsion precipitates, dried products thereof, or combinations thereof is isolated from the by-products. Then, at least a portion of the emulsifier is recycled to one or more of the saccharification, the fermentation, the distillation, and/or the separation to enhance recovery of the vegetable oil.

In other approaches or embodiments, the method may be combined with one or more optional features or method steps in any combination. In such approaches or embodiments, the methods may further include one or more the following: wherein the emulsifier includes at least a protein of at least one of globulin-1 S allele precursor, globulin-1, globulin 2 precursor, globulin-1 S allele-like, oleosin Zm-I, oleosin Zm-II, zeamatin-like protein, granule-bound starch synthase, trypsin inhibitor, and gamma zein (16 kDa), and mixtures thereof; and/or wherein the emulsifier is an oil emulsion and includes at least a protein of Globulin-1 S allele precursor, Globulin-1, Globulin 2, Oleosin Zm-I, Oleosin Zm-II, Zeamatin-like protein, Granule-bound starch synthase, Trypsin inhibitor, gamma zein, or mixtures thereof; and/or wherein the emulsifier is a defatted emulsion precipitate and includes at least a protein of Globulin-1 S allele precursor, Globulin-1, Globulin 2, Globulin-1 S allele-like, Oleosin Zm-I, Oleosin Zm-II, Trypsin inhibitor, Zeamatin-like protein, Granule-bound starch synthase, or combinations thereof; and/or wherein the emulsifier further includes at least one of minerals, modified lipids produced during fermentation, and/or non-germ protein; and/or wherein the emulsifier is a by-product of a bio-refinery process obtained from a bio-refinery process stream selected from the group consisting of beer, whole stillage, thin stillage, and syrup; and/or wherein the saccharification and fermentation occur simultaneously; and/or wherein the recycling is introduced into one or more of a slurry stream, a fermentation vessel, a beer well, and a stillage stream; and/or wherein the emulsifier includes (a) less than about 10% protein, preferably about 1% to about 5% protein, (b) about 30% to about 60% gain oil, and (c) about 40% to about 60% water, and/or wherein the emulsifier is dried and includes (a) about 2 to about 15% protein, (b) about 60% to about 85% gain oil, and (c) less than about 10% water, preferably about 0.5% to about 10% water; and/or wherein the emulsifier is defatted and includes (a) about 2 to about 15% protein, (b) about 5% to about 20% gain oil, and (c) about 60% to about 90% water; and/or wherein the emulsifier is dried and defatted and includes (a) about 10 to about 60% protein, preferably about 10 to about 40% protein, or preferably about 30 to about 60% protein, (b) about 20% to about 45% gain oil, preferably about 20% to about 40% grain oil, and (c) less than about 15% water, preferably about 0.5% to about 15% water; and/or wherein the emulsifier is a precipitate or defatted precipitate and includes and includes (a) about 30 to about 60% protein, (b) about 5% to about 20% gain oil, and (c) less than about 15% water, preferably about 0.5% to about 15% water.

In other approaches or embodiments, the emulsifier is combined with a process stream of the biorefinery and forms an emulsion for enhancing the corn oil yield in a bio-refinery fermentation process. In aspects, the emulsion includes at least a process stream from the bio-refinery fermentation and including at least one of a saccharification stream, a fermentation stream, a beer stream, a whole stillage stream, a wet cake stream, a thin stillage stream, a syrup stream, or combinations thereof combined with an emulsifier in the form of emulsions, defatted emulsions, emulsion precipitates, defatted emulsion precipitates, dried products thereof, or combinations thereof isolated from by-products of the bio-refinery fermentation.

In yet other embodiments, the emulsion described in the previous paragraph may further include other embodiments or features in any combination. These other features or embodiments of the emulsion may include one or more of the following: wherein the emulsifier includes at least a protein of at least one of globulin-1 S allele precursor, globulin-1, globulin 2 precursor, globulin-1 S allele-like, oleosin Zm-I, oleosin Zm-II, zeamatin-like protein, granule-bound starch synthase, trypsin inhibitor, and gamma zein (16 kDa), and mixtures thereof; and/or wherein the emulsifier is an oil emulsion and includes at least a protein of Globulin-1 S allele precursor, Globulin-1, Globulin 2, Oleosin Zm-I, Oleosin Zm-II, Zeamatin-like protein, Granule-bound starch synthase, Trypsin inhibitor, gamma zein, or mixtures thereof; and/or wherein the emulsifier is a defatted emulsion precipitate and includes at least a protein of Globulin-1 S allele precursor, Globulin-1, Globulin 2, Globulin-1 S allele-like, Oleosin Zm-I, Oleosin Zm-II, Trypsin inhibitor, Zeamatin-like protein, Granule-bound starch synthase, or combinations thereof; and/or wherein the emulsifier further includes at least one of minerals, modified lipids produced during fermentation, and/or non-germ protein; and/or wherein the emulsifier is a by-product of a bio-refinery process obtained from a bio-refinery process stream selected from the group consisting of beer, whole stillage, thin stillage, and syrup; and/or wherein the saccharification and fermentation occur simultaneously; and/or wherein the isolated emulsifier is recycled and introduced into one or more of a slurry stream, a fermentation vessel, a beer well, and a stillage stream of the bio-refinery process; and/or wherein the emulsifier includes (a) less than about 10% protein, preferably about 1% to about 5% protein, (b) about 30% to about 60% gain oil, and (c) about 40% to about 60% water; and/or wherein the emulsifier is dried and includes (a) about 2 to about 15% protein, (b) about 60% to about 85% gain oil, and (c) less than about 10% water, preferably about 0.5% to about 10% water; and/or wherein the emulsifier is defatted and includes (a) about 2 to about 15% protein, (b) about 5% to about 20% gain oil, and (c) about 60% to about 90% water; and/or wherein the emulsifier is dried and defatted and includes (a) about 10 to about 60% protein, preferably about 10 to about 40% protein, or preferably about 30 to about 60% protein, (b) about 20% to about 45% gain oil, preferably about 20% to about 40% grain oil, and (c) less than about 15% water, preferably about 0.5% to about 15% water; and/or wherein the emulsifier is a precipitate or defatted precipitate and includes (a) about 30 to about 60% protein, (b) about 5% to about 20% gain oil, and (c) less than about 15% water, preferably about 0.5% to about 15% water.

### BRIEF DESCRIPTION OF THE DRAWINGS

FIG. 1 is a schematic block diagram of an embodiment of processing a grain feedstock to form thin stillage and wet cake;

FIG. 2 is a schematic diagram of embodiments to further process the thin stillage or wet cake to form emulsions and defatted emulsions;

FIG. 3 is a schematic diagram of an embodiment that further processes emulsions and defatted emulsions to obtain emulsifiers or fractions thereof suitable for recycle within a bio-refinery to enhance corn oil recovery; and

FIGS. 4 to 8 are graphs of corn oil recovery amounts using the methods of the present disclosure.

### DESCRIPTION

Industrial fermentation involves the breakdown of a feedstock by a micro-organism, such as yeast and/or bacteria, into ethanol and one or more by-products including distillers corn oil. A traditional ethanol fermentation process utilizes grain-based feedstocks (such as corn, sorghum/milo, barley, wheat, etc.) or other sugar sources (such as sugar cane, sugar beets, etc.) to produce the ethanol and the one or more fermentation by-products. In a typical ethanol plant, however, corn is most often used as the feedstock and ethanol is produced by fermentation of the corn. Enzymes, whether endogenous to the grain, added to the fermenter, or produced by yeast, convert components of the feedstock into simple sugars. Microorganisms, acting subsequent to or simultaneously with the enzymes, convert the simple sugars to ethanol and carbon dioxide. Similar fermentations can be carried out on a variety of feed stocks utilizing a variety of organisms to produce a variety of chemicals and by-products.

The fermentation product also includes water and soluble components as well as residual unfermented particulate matter (among other things). The fermentation product is distilled and dehydrated to recover ethanol. The residual matter (whole stillage) including water, soluble components, oil, and unfermented solids can be further processed to separate out desirable fermentation by-products. Certain components released from the feedstock, such as corn, after saccharification and fermentation or even simultaneous saccharification and fermentation (SSF) have been found to form a stable emulsion (or include unique emulsifier components) and can have the same or better utility as other emulsion formulations or emulsifiers. Such emulsions or emulsifiers can be recovered, fractionated as needed, and recycled in the fermentation process to enhance the recovery of oil.

As such, provided herein are methods, compositions, and uses for by-products obtained from large scale bio-refinery operations for production of a target chemical (such as food, fuel additives, pharmaceuticals, or ethanol) and by-products thereof (such as dried distiller's grain or corn oil) produced by fermenting a feedstock with an organism (yeast and/or bacteria). Exemplary feedstocks include any biomass comprised of sugars such as cereal grains, trees, crop residues, etc., including sugars from cane, beets, etc. The methods herein recover and/or isolate emulsions, emulsifier components, and/or fractions thereof that contain emulsifiers and/or components from the fermentation that have emulsive activity and recycle such stream(s) within the bio-refinery for enhanced oil recovery.

In some embodiments, the emulsions, emulsifiers, or emulsifier fractions are derived from a fermentation product, or beer. Fermentation products can be produced by hydrolyzing materials containing sugar polymers and oil to produce a fermentable material containing fermentable sugars and oil and converting the sugars into a fermentation product using an organism capable of fermentation. For example, a grain containing starch and oil may be ground and the starch hydrolyzed into fermentable sugars, e.g., by using one or more enzymes, chemicals, heat, and/or other catalyst. The fermentable sugars may be converted into a target chemical such as an alcohol (ethanol) using yeasts and other microorganisms. The fermentation product can include the target chemical (ethanol), water, oil, additional soluble components, unfermented particulate matter, and the like. The fermentation product can then be distilled to recover the target chemical (ethanol) leaving the remaining components as whole stillage.

A fermentation by-product containing oil can be derived from the one or more grain materials. A variety of grain materials (some of which may also be referred to as vegetable materials) can be used such as whole ground grain or a fraction of a grain. Grain material can be derived from grain such as corn, sorghum, wheat, rice, barley, soybean, rapeseed, oats, millet, rye or any other grains that that are capable of being fermented and subjected to the refined oil process described herein.

In some embodiments, oil in the fermentation product can also be derived from oleaginous microorganisms. Exemplary oleaginous microorganisms include oleaginous microalgae, which can include the genus Chlorella or Prototheca, including, Chlorella protothecoides or Prototheca moriformis, Nannochloropsis salina, Chlorella vulgaris, Scenedesmus dimorphus, and Chaetoceros gracilis. Other exemplary oleaginous microorganisms include yeast such as Yarrowia hpolytica, Cryptococcus curvatus, Rhodosporidium toruloides, and bacteria such as Rhodococcus opacus.

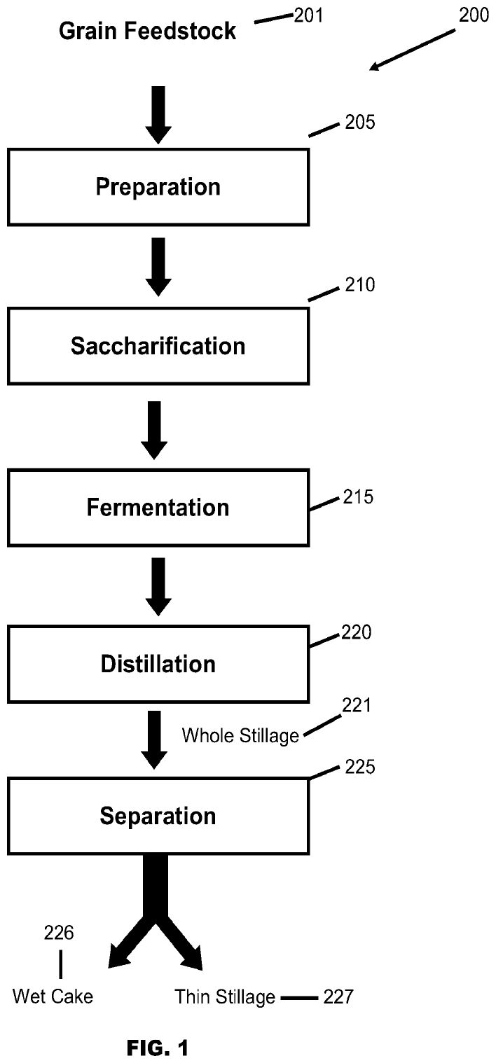

For illustration purposes, an exemplary process for obtaining a corn oil composition from corn grain is described herein. The process includes preparing the corn, saccharifying sugar polymers to obtain fermentable sugars, fermenting the sugars, recovering a corn oil composition feedstock, and refining the corn oil composition feedstock to form a corn oil product. A non-limiting example of providing a corn grain oil composition for refining according to the present disclosure is illustrated in FIGS. 1 to 3 as discussed more below.

Preparation of Grain for Saccharification: As shown in FIG. 1, process 200 includes providing grain feedstock 201 (corn) that is first prepared 205 for saccharification 210 by reducing the size of the grain. In some embodiments, corn grain can be dry milled or hammer milled to produce whole ground corn having a medium-to-fine grind for use in saccharification. In some embodiments, corn grain can be dry-fractionated to separate components of the corn grain (e.g., germ) from each other and then recombine two or more components (e.g., the endosperm and germ) for saccharification.

In some embodiments, the corn grain can be ground so that a substantial portion (in approaches, a majority) of the ground corn grain fits through a sieve with a 0.1 to 5.0 mm screen, or even a 0.1 to 0.5 mm screen. For example, in an embodiment, about 70% or more, of the ground corn can fit through a sieve with a 0.1 to 0.5 mm screen.

Ground corn can be mixed with an appropriate amount of water to form an aqueous composition or slurry for subsequent saccharification of the slurry and fermentation of the resulting sugars. In an embodiment, whole ground corn can be mixed with liquid at about 20 to about 50 wt-% or about 25 to about 45 wt-% dry whole ground corn. The whole ground corn can include starch, fiber, protein, oil, endogenous enzymes, amino acids, etc. Any corn grain components (e.g., residual fiber, starch, sugar, oil, etc.) remaining after fermentation can be extracted/separated after fermentation and/or distillation, as discussed below. Because starch constitutes the largest mass portion of the corn grain it can be more efficient to extract other components (e.g., oil, fiber, protein, etc.) after at least a portion of the starch has been removed (i.e., hydrolyzed into glucose which is consumed by, e.g., yeast).

Saccharification: After forming an aqueous slurry that includes the corn material from preparing corn as described above, the aqueous slurry can be subjected to saccharification 210 to break down (hydrolyze) at least a portion of the starch into glucose that can be used by yeast during fermentation. Saccharification can be performed by a variety of techniques. For example, heat and/or one or more enzymes can be used to saccharify components of the prepared corn into oligomers and monomers.

In some embodiments, a relatively low temperature saccharification process involves enzymatically hydrolyzing at least a portion of the starch in the aqueous slurry at a temperature below starch gelatinization temperatures, so that saccharification occurs directly from the raw native insoluble starch to soluble glucose while bypassing conventional starch gelatinization conditions. Starch gelatinization temperatures are typically in a range of 57° C. to 93° C. depending on the starch source and polymer type. Converting raw starch to glucose with one or more exogenous enzymes, e.g., glucoamylase and acid fungal amylase is described in U.S. Pat. No. 7,842,484 (Lewis) and U.S. Pat. No. 7,919,291 (Lewis et al.), wherein the entireties of the patents are incorporated herein by reference. In one embodiment, saccharification includes enzymatically (e.g., with alpha-amylases and gluco-amylases) hydrolyzing at least a portion of the starch in the aqueous slurry at a temperature below 40° C. or less to produce a slurry that includes glucose. In some embodiments, enzymatic hydrolysis occurs at a temperature in the range of from 25° C. to 35° C. to produce a slurry that includes glucose.

In some embodiments, saccharification of starch can include heating the slurry to a temperature in the range from 50° C. to 100° C.; from 60° C. to 90°; or even from 80° C. and 85° C. and adding a thermostable alpha-amylase to the slurry to initiate liquefaction. In some embodiments, saccharification of the starch can include jet-cooking the slurry at a temperature between 100° C. to 145° C. to complete gelatinization of the slurry.

Fermentation: After saccharification, the resulting slurry (grain mash composition) includes grain solids, grain oil and sugar. The sugar (glucose) that is generated from saccharification can be fermented 215 into one or more biochemicals (butanol, ethanol, and the like). Systems for producing more than one biochemical from the glucose can be integrated together or be separate. Fermenting can be carried out by microorganisms. Exemplary microorganisms include ethanologens, butanologens, and the like. Exemplary microorganisms include yeasts.

In some embodiments, fermenting can include contacting an aqueous slurry including sugars derived from ground corn with microorganisms under conditions suitable for growth of the microorganims and production of a biochemical. For examples, yeasts may be used that convert the sugars to ethanol. Suitable yeasts include any variety of commercially available yeasts, such as commercial strains of Saccharomyces cerevisiae. Optionally, one or more components (e.g., yeast nutrients) can be included in the aqueous slurry that is to be fermented. In other embodiments, saccharification and fermentation can occur simultaneously in the same reactor (also referred to as simultaneous saccharification and fermentation (SSF)). In yet other embodiments, fermenting a grain mash can include fermenting the grain mash in the presence of one or more enzymes (endogenous enzymes and/or exogenous enzymes) to generate one or more fatty acid alkyl esters. Examples of such enzymes include lipase, esterase, and combinations thereof.

Distillation: After fermentation, the biochemical can be removed from the beer in a distillation system 220 to form a whole stillage 221. For example, a beer derived from corn can be distilled to remove ethanol and form whole stillage. For example, heat and/or vacuum may be applied to the fermentation product in a distillation unit to evaporate and condense the biochemical to separate it from the rest of the fermentation product. The bottoms stream from the distillation unit after the biochemical has been recovered is referred to as whole stillage 221. This whole stillage stream 221 includes, e.g., suspended solids, dissolved solids, water, and oil. The whole stillage stream is separated, typically by decanting centrifuges, into a thin stillage stream 227 and a wet cake stream 226. The wet cake stream 226 is a wet, solid stream e.g. greater than 25% solids w/w. The thin stillage stream 227 is a liquid stream that contains a lower concentration of suspended solids, e.g. less than 15% solids w/w, compared to whole stillage.

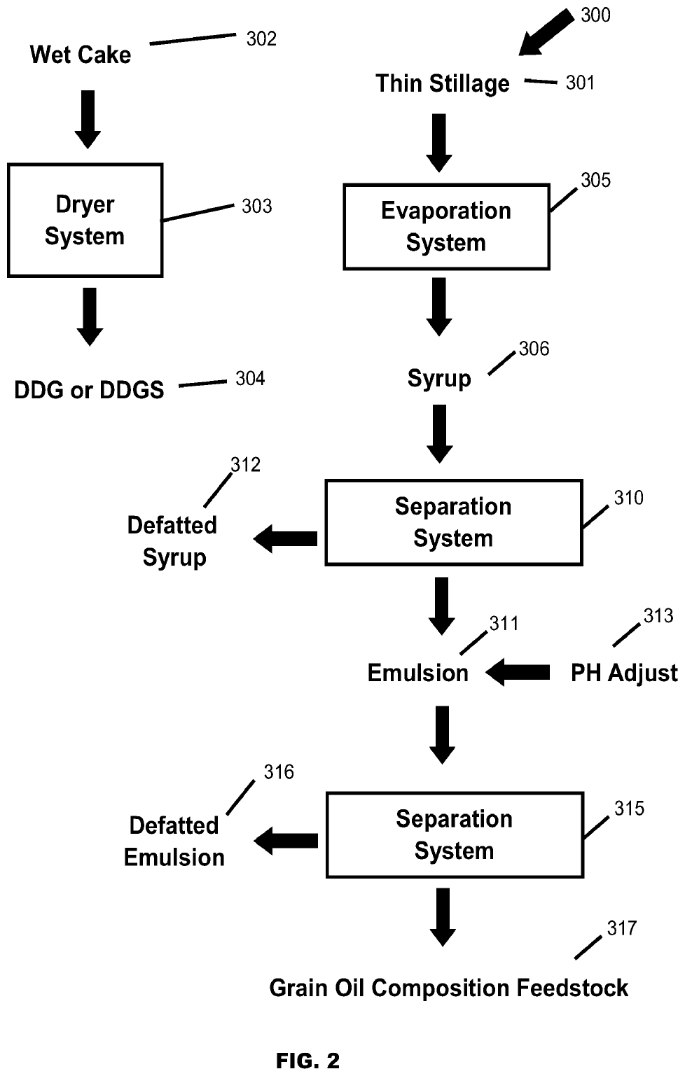

Turning to FIG. 2, non-limiting, exemplary process flow 300 illustrates further processing of the thin stillage stream 301 and/or the wet cake stream 302 obtained from the processes of FIG. 1. The solids concentration of the thin stillage stream can be increased in an evaporation step 305 where water is evaporated from the thin stillage 301. Concentrated thin stillage is referred to as syrup 306 in the art. The syrup stream 306 contains an increased concentration of corn oil, which can be separated as an oil composition and sold as distiller's corn oil (DCO). The wet cake 302 can be dried in a dryer system 303 to provide “Distillers Dried Grains” (DDG) 304. In some embodiments, a portion of the syrup may be blended into DDG or added to the wet cake before drying to produce Distillers Dried Grain with Solubles (DDGS).

The syrup 306 can be processed to provide distiller's grain oil and other by-products including the emulsifiers herein. As shown in FIG. 2, the syrup can be separated via separation system 310 into a first oil fraction or emulsion 311 and a first aqueous fraction 312 (defatted syrup). The first oil fraction 311 can be referred to as distiller's corn oil or further processed as discussed more below. As shown in FIG. 2, if first oil fraction 311 is an emulsion, it can have its pH adjusted (e.g., from 7-9) to break the emulsion into a second oil fraction 317 and a second aqueous fraction 316 (defatted emulsion). The second oil fraction 317 can also be referred to as distiller's corn oil.

Examples of methods of extracting oil from a stillage composition are described at U.S. Pat. No. 9,061,987, (Bootsma), U.S. Pat. No. 8,702,819 (Bootsma), and U.S. Pat. No. 9,695,449 (Bootsma) wherein oil is separated using centrifuges. The entireties of these patents are incorporated herein by reference. U.S. Pat. No. 8,008,516 (Cantrell et al.) describes DCO separation from thin stillage, wherein the entirety of the patent is incorporated herein by reference. U.S. Pat. No. 9,896,643 (Redford) describes recovering a light phase product from ethanol product, wherein the entirety of the patent is incorporated herein by reference.

Optionally, a grain oil composition feedstock can be treated before being refined according to the present disclosure. Non-limiting examples of such treatments include one or more of degumming, adding a flocculating agent to the grain oil composition, adding a filter aid to the grain oil composition.

As discussed more below, the emulsion 311 or the defatted emulsion 316 contain natural emulsifiers obtained as a by-product of the fermentation. Those emulsions, emulsifiers therein, components thereof with emulsive activity, and/or fractions thereof may be recovered, isolated, and/or recycled within the bio-refinery process to enhance the yield of corn oil from the process.

Oil emulsions consist of two immiscible liquids where liquid droplets of one polarity are dispersed in a liquid continuous phase of a different polarity. Emulsions are typically formed by high shearing oil, water, and emulsifiers together. High shearing is accomplished by means such as simple pipe flow, static mixers, colloid mills, high-pressure homogenizers, and ultrasound generators. Considerable effort is made to produce stable emulsions that do not break down prematurely in use. Emulsifiers are used to stabilize emulsions by increasing the kinetic stability of an emulsion. Emulsifiers typically have a polar or hydrophilic part and a non-polar (i.e. hydrophobic or lipophilic) part. Some emulsifiers are more soluble in water and generally form oil-in-water emulsions, while other emulsifiers are more soluble in oil and form water-in-oil emulsions. Surfactants are exemplary emulsifiers.

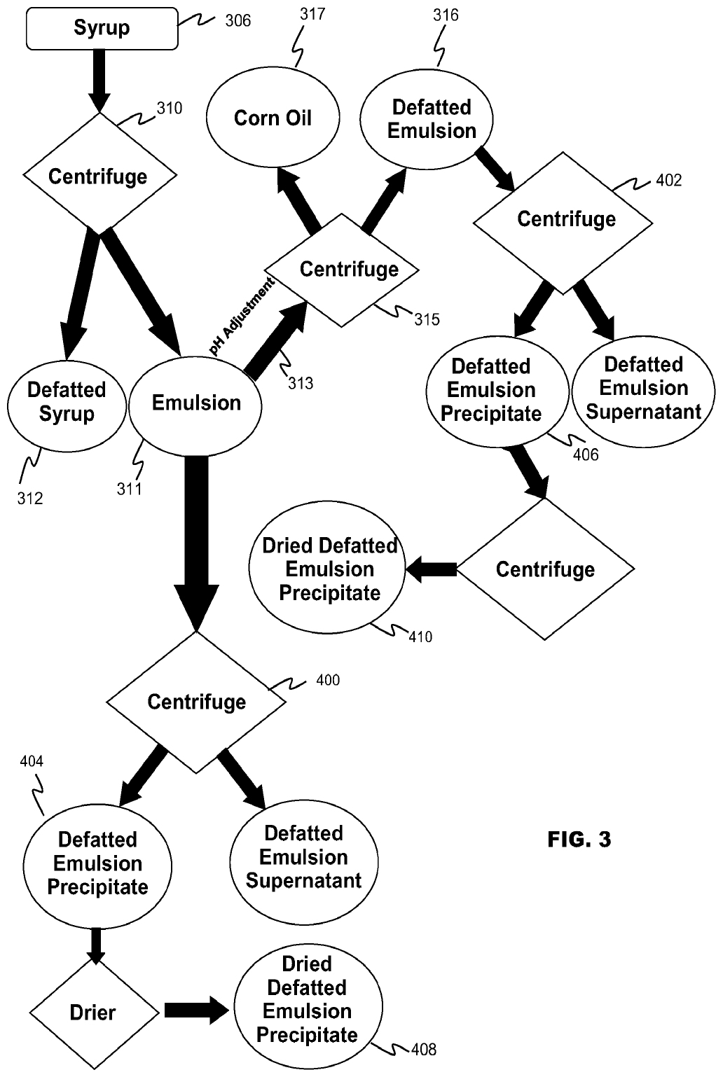

Turning to FIG. 3, further processing of the emulsion 311 or the defatted emulsion 316 is shown to obtain emulsifiers including emulsions, defatted emulsions, defatted emulsion precipitates, and/or fractions or dried products thereof (i.e., powders) provided herein as by-products of a bio-refinery process that are suitable for being recycled within the bio-refinery to enhance the yield of oil within the process. The emulsion 311 (or a fraction/dried product thereof as a centrifuge precipitate or pellet), the defatted emulsion 316 (or a fraction/dried product thereof as a centrifuge precipitate or pellet) is recovered or isolated and then recycled within the bio-refinery to one or more of the saccharification 201, the fermentation 215, the distillation 220, and/or the various separation steps 221, 225, 226, and/or 227 of FIG. 1 to enhance the yield of corn oil 317 recovered from the process.

The emulsifiers including the emulsions, defatted emulsions, defatted emulsion precipitates, and/or dried products thereof include components, such as proteins and at least one non-germ proteins, that have been found to enhance the yield and/or increase the amount of oil recovered from the processes herein when recycled within the bio-refinery. Without wishing to be limited by theory, it is believed that the emulsions 311 or defatted emulsion 316 or fractions thereof obtained by the further processing set forth in FIG. 3 include components with emulsive functionality and, when recovered or isolated and then recycled within the bio-refinery as discussed above, enhance the recovery of oil by extracting more oil and/or strengthening the affinity to oil enabling the transfer or removal of oil from the solids or other components within the various processing streams. Such recycling of these streams would not have been expected to increase oil yield due, in part to, to the presence of oil already within the recycled streams and/or the solids content of such streams. However, as shown in the Examples below, when such emulsive streams are recycled, the oil recovered is enhanced above and beyond any oil that may be contributed by the recycle streams. Without wishing to be limited by theory, the emulsifiers, emulsions, or defatted emulsions herein are enriched in corn or corn-derived proteins. These proteins are amphiphilic, meaning they contain both polar and non-polar surfaces. Non-polar surfaces tend to interact with the oil phase and polar surfaces tend to interact with the aqueous phase. The corn proteins can adsorb at the interface of oil droplets to hinder their coalescence. The charge or polarization of these protein residues are dependent upon pH and potential interaction with minerals or lipid bodies. Such characteristics and/or interactions are believed to be one factor that may aid in the enhancement of oil recovery in the methods herein.

In one approach, for example, emulsions herein may have protein in amounts of less than about 10%, or less than about 7%, or less than about 5%, or about 1% to about 5%, about 2% to about 4%, or about 1% to about 3% of the emulsion (or other ranges between the noted protein amounts). The emulsion also include about 30% to about 60% grain oil, for example, about 30% to about 50% grain oil, or about 40% to about 60% grain oil, or about 30% to about 40% grain oil, or about 50% to about 60% grain oil. Water can be present in the emulsion in an amount from about 40% to about 60%, or about 40% to about 50%, or about 50% to about 60% water.

An exemplary emulsion is a corn oil emulsion from SSF via dry-grind ethanol production. The emulsion may include emulsifier components generated by the SSF of corn that are unique due to the formation of and integration of proteins including non-germ proteins (discussed more below), minerals, and/or modified lipids, etc. from the SSF. For instance, the modified lipids may include glycerides, free fatty acids, and alkyl esters (i.e., fatty acid ethyl esters, etc.) derived from the fermentation of corn and/or the SSF of corn. The various forms of the emulsifier herein may also include up to about 10,000 ppm of minerals. Exemplary minerals may include one or more of calcium (40 to 100 ppm), copper (1 to 10 ppm), iron (10 to 20 ppm), magnesium (500 to 1000 ppm), manganese (1 to 10 ppm), phosphorus (1000 to 3000 ppm), potassium (1500 to 4000 ppm), sodium (300 to 1000 ppm), sulfur (1000 to 3000 ppm), and/or zinc (1 to 20 ppm). The resulting emulsions 311, defatted emulsions 316, precipitates, and/or dried products thereof contain a high concentration of emulsifier components derived from the SSF of corn including the proteins, the lipids, the modified lipid products, the minerals, and/or starch complexes that can further be used to emulsify non-miscible materials.

While not wishing to be held to theory, the corn oil based emulsions 311, defatted emulsions 316, precipitates, and/or dried products thereof contain specific proteins that contribute to emulsibility and corn oil yield enhancement when recycled within the bio-refinery. In addition, other non-protein elements are possibly modified during ethanol production and may contribute to emulsibility and corn oil yield enhancement. It has also been determined that the emulsion is unstable at pH values from about 7.0 to about 8.5. The concentration of the various emulsifier components derived from the SSF of corn for example (such as proteins, minerals, carbohydrates, lipids, and modified lipids) present in the emulsion, defatted emulsion, precipitates, and/or dried products thereof can affect how the emulsifiers perform in various applications. Tables 2 to 6 in the Examples below provide exemplary compositional analyses of emulsions 311 or defatted emulsions 316 herein.

An emulsifier disclosed herein can be in the form of an emulsion, defatted emulsion, precipitates and/or dried products thereof (such as powders) as shown in FIG. 3. The emulsion, defatted emulsion, precipitate, and/or dried products thereof can comprise (a) protein including at least one non-germ protein; (b) grain oil; and (c) water, also referred to herein as moisture, and is a by-product of a biorefinery process, for example, corn ethanol fermentation. In some embodiments, the emulsion comprises (a) less than about 10 percent protein or from about 1% to about 10% protein or about 1 to about 5% protein including at least one non-germ protein, (b) from about 30% to about 60% grain oil, and (c) from about 40% to about 60% water, wherein the emulsion is a by-product of a biorefinery process. The emulsion preferably contains no detectable phospholipids and/or less than about 1% polymerization. The emulsion can include minerals and modified lipids produced during fermentation as discussed above.

The protein present in the emulsifiers herein, such as the emulsions, defatted emulsions, precipitates, and/or dried products thereof, can be sourced from a biorefinery process source stream, such as from a dried protein stream or a concentrated protein stream. The protein can be sourced from an aqueous stream separated from a biorefinery process stream. In one approach or embodiment, the protein(s) may be from protein families including globulin protein, heat shock protein, oleosins, peptidases, or combinations thereof. In other approaches or embodiments, the at least one protein can be globulin-1 S allele precursor, globulin-1, globulin 2 precursor, globulin-1 S allele-like, oleosin Zm-I, oleosin Zm-II, zeamatin-like protein, granule-bound starch synthase, trypsin inhibitor, and gamma zein (16 kDa), or mixtures thereof.

The emulsifier can be dried, i.e. part of or most of the moisture can be removed. An exemplary dried emulsifier may be a dried emulsion product obtaining by drying the emulsion and comprises (a) from about 2% to about 15% protein, (b) from about 60% to about 85% grain oil, and (c) less than about 10 percent water or from about 0.5% to about 10% water.

The emulsifier can be defatted, i.e. part of or most of the oil can be removed. An exemplary defatted emulsifier may be a defatted emulsion and comprises (a) from about 2% to about 10% protein on a dry weight basis; (b) from about 5% to about 20% grain oil on a dry weight basis; and (c) from about 60% to about 90% water.

The emulsifier can be dried and defatted. An exemplary dried and defatted emulsifier may be obtained by drying the defatted emulsion (preferably to form a powder) and comprises (a) from about 10% to about 40% protein on a dry weight basis; (b) from about 20% to about 45% grain oil on a dry weight basis, and (c) a moisture content of less than about 15%. In some aspects, the emulsifier is a shelf-stable emulsifier either by further removing water or adding additional components. For instance, shelf-stable emulsifier may obtained by drying the defatted emulsion and comprises (a) from about 30% to about 60% protein, (b) from about 20% to about 40% grain oil, and (c) less than about 15% water, for example, a moisture content of about 0.5% to about 15%. In some aspects, the shelf stable emulsifier may further include an optional antimicrobial and/or an optional antioxidant. Exemplary antioxidants include TBHQ, ethoxyquin, etc. Exemplary antimicrobials include phenoxyethanol, quaternary ammonium compounds, isothiazolinones, etc.

An exemplary dry defatted emulsifier from a grain-to-ethanol biorefinery is obtained from drying a defatted emulsion and comprises (a) about 30% to about 60% protein on a dry weight basis; (b) about 20% to about 40% grain oil on a dry weight basis; and (c) a moisture content of about 0.5% to about 15%. The dry defatted emulsifier can be obtained from an aqueous stream separated from a biorefinery process stream. The biorefinery process stream can be selected from the group consisting of beer, whole stillage, thin stillage, and syrup. The dry defatted emulsifier can be reconstituted prior to use, or can be added directly to a substance to form an emulsion.

The emulsifier can be a precipitate or a defatted precipitate (otherwise referred to as a centrifuge pellet). An exemplary defatted and precipitated emulsifier may be a dried and emulsion precipitate and comprises (a) from about 30% to about 60% protein on a dry weight basis; (b) from about 5% to about 20% grain oil on a dry weight basis; and (c) less than about 15% water.

Table 1 provides exemplary moisture, protein, fat, and ash ranges on a % w/w basis for emulsifiers suitable for being recycled within a bio-refinery in various emulsion, precipitate, and/or dried product (i.e., powder) forms.

TABLE 1
Moisture, Protein, and Oil Ranges for Exemplary Emulsions

| | % w/w Range (Typical) | | | |
|---|---|---|---|---|
| Material | Moisture | Protein | Fat | Ash |
| Emulsion | 30-60 (35) | 1-5 (3) | 30-60 (50) | 2-10 (5) |
| Dried Emulsion | ≤10 (5) | 2-15 (5) | 60-85 (75) | 5-20 (10) |
| Defatted Emulsion | 60-90 (70) | 2-10 (6) | 5-20 (10) | 2-15 (7) |
| Dried Defatted Emulsion | ≤15 (5) | 10-40 (20) | 20-45 (30) | 10-35 (20) |
| Dried Defatted Emulsion | ≤15 (5) | 30-60 (45) | 20-40 (30) | 5-20 (10) |
| Precipitate | | | | |

### Methods of Fractionating Emulsions

The emulsion 311 or defatted emulsion 316 obtained from the methods of FIGS. 1 and 2 may be recycled within the bio-refinery “as is” to enhance oil recovery, but may also be further fractionated to isolate fractions or portions thereof having emulsive activity prior to being recycled within the bio-refinery. The isolated fractions may be a filtrate, precipitate, and/or a centrifuge pellet. Such fractionation is exemplified in FIG. 3 by the further centrifuge steps 400 or 402 by centrifuging the emulsion 311 and/or the defatted emulsion 316 to produce an emulsion precipitate 404 or a defatted emulsion precipitate 406 (in either instance, often called a centrifuge pellet). Without wishing to be limited by theory, it is believed that such precipitates/pellets 404 and/or 406 may have enhanced levels of components, such as those discussed above and shown in the Examples, with emulsive activity that lead to the enhanced corn oil recovery when recycled within the bio-refinery.

While the further fractionation of FIG. 3 is shown via a centrifuge 400 or 402, other separation methods may also be used as needed including, filtering, gravity phase separation to occur over time using a settling tank and/or a cooled settling tank, followed by decanting the oil phase layer. However, in some embodiments fractionation or separation is accomplished more quickly by centrifugation. These and other methods may be combined. Centrifugation can be by, for example, a decanter centrifuge, a disk stack centrifuge, a cooled disk stack centrifuge, a screen centrifuge, hydrocyclone or a combination thereof. The speed or amount of centrifugal force to recovery the precipitate or pellet applied can depend on various factors such as sample size and may be adjusted appropriately depending on such factors. For example, centrifugation may be carried out at about 4,000 to about 5,000 rpm or about 4,200 rpm. In some embodiments, centrifugation is carried out at about 4,200 rpm, for about 20 minutes and at about 27° C. Non-limiting examples of other apparatuses that can be used to separate the precipitate or pellet may include a filter press, a rotary drum filter, or some other apparatus that is suitable to separate a liquid stream based on density differences. Any method, apparatus for such separation may be combined as needed for a particular application.

If desirable, the emulsion 311, the defatted emulsion 316, or the precipitates 404 and/or 406 can be dried to form dried products. For example, the methods can further isolate or separate components by drying such process steams to a moisture content of less than about 15%, less than 10%, or 0.5 to 10% to produce a dried emulsion product, a dried defatted emulsion product, a dried emulsion precipitate product 408, or a dried and defatted emulsion precipitate product 410. Preferably, dried products are in the form of a powder, which can be reconstituted prior to recycle if needed

### Emulsifier Recycling

The emulsion 311, the defatted emulsion 316 or fractions or dried products thereof such as the precipitates/pellets 404 and 406 or the dried products thereof 408 and 410 are recovered and/or isolated and then recycled within the bio-refinery to enhance downstream corn oil recovery 317. As noted above, the recycling of such streams may be to one or more of the saccharification step 201, the fermentation step 215, the distillation step 220, and/or the various separation steps 221, 225, 226, and/or 227 of FIG. 1 to enhance the yield of corn oil 317 recovered from the process or corn oil recovered from other by-products of the process.

Recycling may be via a continuous process or may be batch-wise as needed for a particular application. The recycling may be in amounts of about 0.5 to about 10 weight percent (in other approaches, about 0.5 to about 5 weight percent) of the isolated/recovered emulsifier streams to the various upstream processing steps. If recycling is to the whole stillage, thin stillage, or wet cake streams, the noted stream and recycled emulsifiers combination may be incubated for a period of time, such as about 1 to about 24 hours, about 4 to about 24 hours, about 4 to about 15 hours, about 4 to about 10 hours, or even about 4 to about 8 hours to allow for the corn oil recovery enhancement. Incubation may proceed with mixing, such as with a mixing speed of about 100 to about 500 rpm or about 250 rpm. Such incubation may be at temperatures of about 25° C. to about 80° C., and in some instances, about 30° C. to about 60° C., or about 30° C. to about 35° C.

If the above described emulsifier recycle is to an ethanol fermentation process, various parameters provided in U.S. Pat. No. 8,702,819 can be used to generate the emulsion or defatted emulsion disclosed herein, and to tailor performance characteristics for a given application as described above. Exemplary process parameters include: process temperature, adjusting the pH used to break the emulsion (breaking at higher pH results in greater release of proteins, versus lower pH), residence time at emulsion breaking pH (composition changes include a change in particle size and free proteins the longer the residence time), centrifuge type, and any other operating parameters (distillation temperature, evaporator temperature, residence time).

### EXAMPLES

The following examples are provided for illustrative purposes only and are not intended to limit the scope of the invention. In these examples, as well as elsewhere in this application, all ratios, parts, and percentages are by weight unless otherwise indicated.

### Example 1

The oil emulsion and defatted emulsion (after pH adjustment and subsequent oil extraction), described herein, differ in multiple ways from other emulsions or oil bodies mentioned in various publications. For example, Majoni et al. (J. Am. Oil Chem. Soc. 88, 425-434, 2011) evaluated different parameters including chemicals, pressure, temperature, and mixing in order to maximize oil recovery from condensed corn distillers solubles (syrup). The authors surmise that at higher pH, proteins are easily solubilized to make a better emulsifier. Indeed, the authors reported higher oil recovery at acidic pH values. However, the oil emulsion described herein is stable at those pH values and becomes unstable at pH values between 7-8.5.

Comparing the most prevalent proteins in (a) the presently described oil emulsion with (b) the defatted emulsion precipitate after pH adjustment to 8.3 (Table 2), demonstrates that increasing the pH causes the proteins to precipitate, thus freeing the oil to be extracted.

TABLE 2
Top 10 Proteins in Oil Emulsion and Defatted Emulsion Precipitate

| Oil Emulsion^{a} | Defatted Emulsion Precipitate^{b} |
|---|---|
| Globulin-1 S allele precursor | Globulin-1 S allele precursor |
| Globulin-1, partial | Globulin-1, partial |
| Globulin 2 precursor | Globulin 2 precursor |
| Oleosin Zm-I | Globulin-1 S allele-like |
| Oleosin Zm-II | Oleosin Zm-I |
| Zeamatin-like protein | Oleosin Zm-II |
| Granule-bound starch synthase | Trypsin inhibitor |
| Trypsin inhibitor | Zeamatin-like protein |
| 16 kDa gamma zein | Granule-bound starch synthase |
| Probable non-specific lipid- | uncharacterized protein |
| transfer protein 2 | LOC100280650 precursor |

^{a}Oil emulsion prior to pH adjustment (pH ≈ 3.8)
^{b}Defatted Emulsion precipitate formed after pH adjustment (pH ≈ 8.3) and isolated via centrifugation

Zayas (Cereal Chem. 66, 263-267, 1989) and Nikiforidis (J. Agric. Food Chem. 57, 5591-5596, 2009) mention the use of germ proteins as emulsifiers to generate emulsions or oil bodies. However, these emulsifiers mentioned in Zayas are exclusively germ proteins and do not include other components of the emulsion described herein such as non-germ proteins, minerals, and modified lipids produced during fermentation. The compositional differences account for the physical difference in the emulsions, most notably the stability at different pH values. For example, Nikiforidis et al. describes the instability of the produced oil bodies when the pH is between 3.8 and 6.5. Similarly, Zayas et al. describes increasing emulsifying capacity and stability with increasing pH especially between 7 and 8.5.

### Example 2

Different samples of an emulsion (1, 2, and 3) were obtained from POET bio-refineries for compositional analysis. The emulsions were analyzed for volatiles by a Mettler Toledo HR83-P Halogen Moisture Analyzer. The oil content of the emulsions was determined gravimetrically via multiple extractions with chloroform. The non-oil solids were determined gravimetrically as the remaining dried solids after oil extraction. The mineral content of the dried solids were determined by Foundation Analytical Laboratory by ICP following microwave digestion. The amino acid profile was determined by Midwest Laboratories on the dried emulsion or dried defatted emulsion according to AOAC 994.12 (Alt. III). Free fatty acid (FFA) content of the emulsion was determined using a Mettler Toledo T70 titrator using diethyl ether and ethanol as the diluent and potassium hydroxide in ethanol as the titrant. See Tables 3-5.

The most abundant proteins in the emulsions were identified by Q exactive HF-X mass spectroscopy. Samples were prepared by first extracting oil from the emulsion using chloroform. Next, the proteins in the defatted emulsion were then solubilized by boiling in 8M urea. The solution was then further diluted 1:10 in 8M urea with the addition of 4% (% w/w) SDS to assist solubilization. Samples were sent to Bioproximity to perform the prep and analysis for identification. Prep and analysis included protein extraction and cleanup, trypsin digestion, solid-phase extraction, and UPLC-MS/MS including Thermo Q-Exactive HF-X quadrupole-Orbitrap mass spectrometry. Only the 10 most abundant proteins are shown in each case. See Table 6.

TABLE 3
Compositional Analysis of Emulsions on As-is Basis

| | | | | Total | |
|---|---|---|---|---|---|
| | % Non- | | % Non- | Elemental | |
| | volatiles | % Oil | oil solids | Minerals | % FFA |
| Sample | (% w/w) | (% w/w) | (% w/w) | (ppm) | (% w/w) |
| Emulsion 1 | 69.7% | 55.2% | 15.3% | 6509 | 7.5% |
| Emulsion 2 | 61.6% | 44.9% | 16.4% | 9289 | 10.2% |
| Emulsion 3 | 44.5% | 29.5% | 14.9% | 8565 | 5.8% |

TABLE 4
Elemental Mineral Analysis of Emulsions on As-is Basis

| Elemental | Emulsion 1 | Emulsion 2 | Emulsion 3 |
|---|---|---|---|
| Mineral | (ppm) | (ppm) | (ppm) |
| Calcium | 46 | 67 | 80 |
| Copper | 1 | 1 | 3 |
| Iron | 12 | 17 | 12 |
| Magnesium | 685 | 905 | 923 |
| Manganese | 3 | 4 | 4 |
| Phosphorous | 1621 | 2156 | 2325 |
| Potassium | 2082 | 3022 | 3060 |
| Sodium | 476 | 814 | 217 |
| Sulfur | 1575 | 2289 | 1932 |
| Zinc | 9 | 12 | 10 |
| Total | 6509 | 9289 | 8565 |

TABLE 5
Amino Acid Profile of Emulsion and
Defatted Emulsion on As-is Basis

| | Emulsion | Defatted Emulsion |
|---|---|---|
| Amino Acid | (% w/w) | (% w/w) |
| Alanine | 0.21% | 0.24% |
| Arginine | 0.21% | 0.19% |
| Aspartic Acid | 0.24% | 0.22% |
| Cystine | 0.10% | 0.10% |
| Glutamic Acid | 0.44% | 0.45% |
| Glycine | 0.18% | 0.16% |
| Histidine | 0.10% | 0.10% |
| Isoleucine | 0.11% | 0.09% |
| Leucine | 0.22% | 0.18% |
| Lysine (total) | 0.15% | 0.14% |
| Methionine | 0.08% | 0.03% |
| Phenylalanine | 0.12% | 0.10% |
| Proline | 0.25% | 0.16% |
| Serine | 0.15% | 0.14% |
| Threonine | 0.09% | 0.07% |
| Tyrosine | 0.10% | 0.10% |
| Tryptophan | 0.03% | 0.03% |
| Valine | 0.17% | 0.15% |
| Total | 2.95% | 2.65% |

TABLE 6
Top 10 Emulsion Proteins by Relative Abundance

| NCBI Reference Sequence | Protein Name |
|---|---|
| NP_001354320.1 | Globulin-1 S allele precursor |
| PWZ58219.1 | Oleosin Zm-II |
| NP_001295419.1 | Globulin 2 precursor |
| NP_001105338.2 | Oleosin Zm-I |
| ACZ74557.1 | Granule-bound starch synthase |
| NP_001142211.1 | Uncharacterized protein LOC100274379 |
| AQL09053.1 | Vicilin-like seed storage protein |
| P05494.1 | ATP synthase subunit alpha |
| XP_008655895.1 | Endochitinase A-like |
| NP_001105409.2 | Alcohol dehydrogenase |

### Example 3

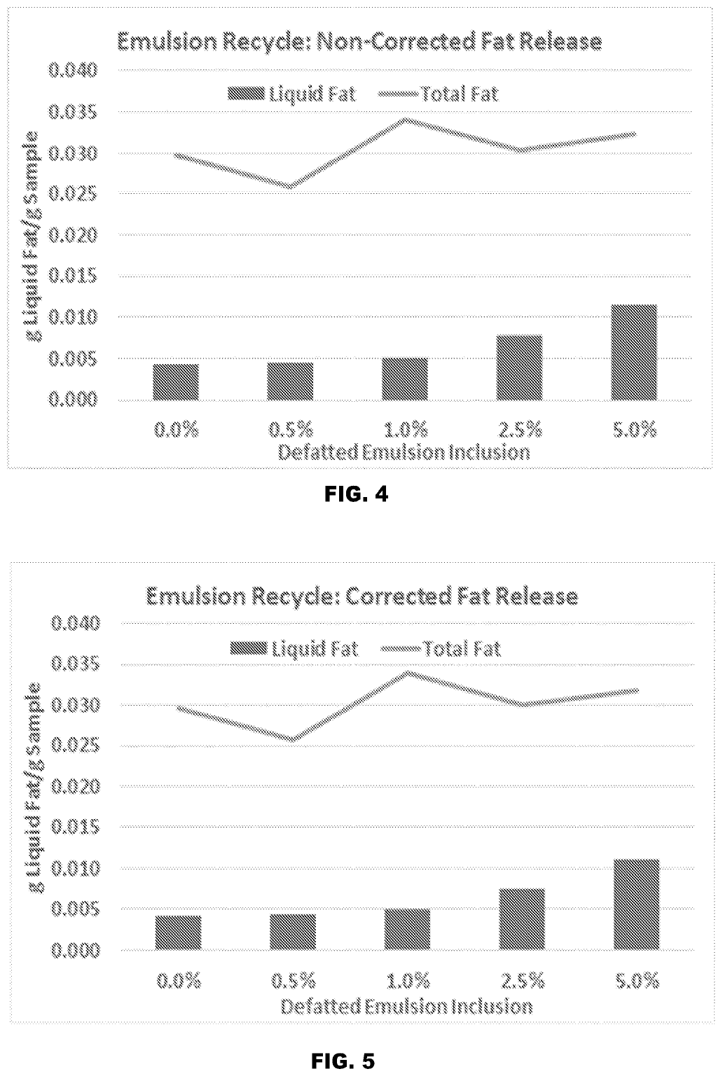

A defatted emulsion product was recycled to an ethanol fermentation step corn bio-refinery at 0% (control), 0.5%, 1%, 2.5%, or 5% of the defatted emulsion in the fermentation. The emulsion Sample had about 1.0% oil. Downstream oil recovery of the samples (i.e., oil 317 of FIG. 2) is shown in FIG. 4 and corrected oil recovery is shown in FIG. 5 (corrected oil recovery subtracts oil added from the recycled emulsion and, thus, accounts only for oil extracted from the grain starting material).

### Example 4

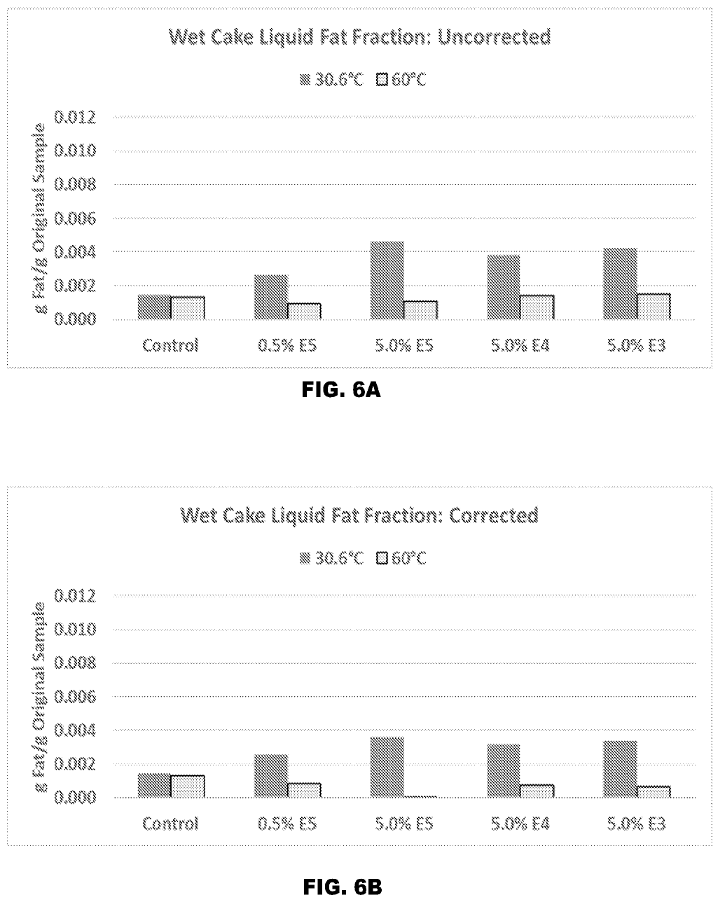

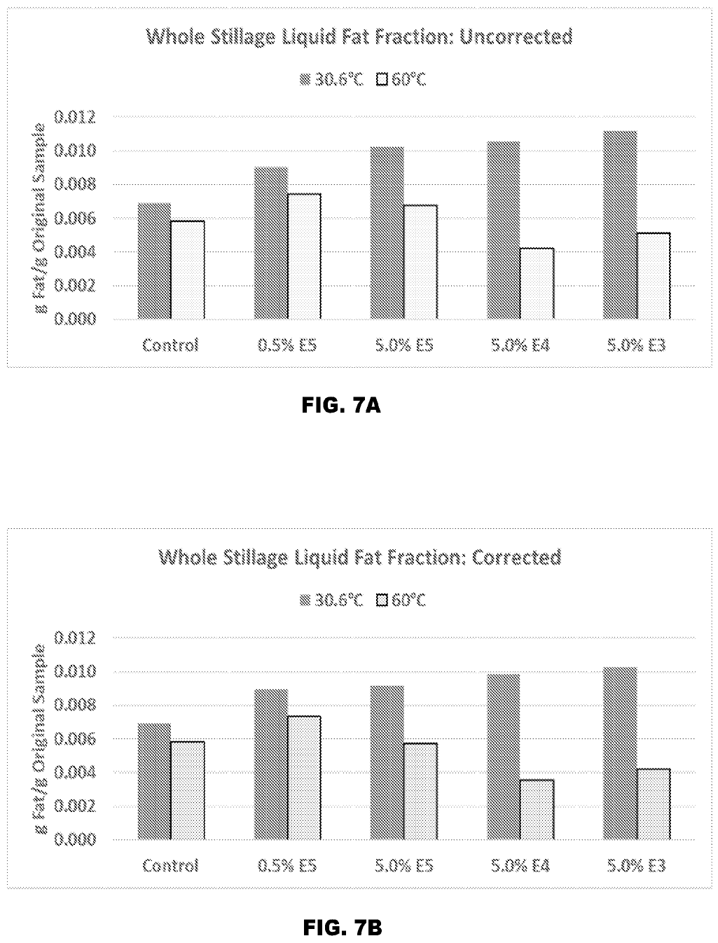

Three different defatted emulsion products (E3, E4, or E5) were recycled to either a whole stillage stream (221) or a wet cake stream (226) in an ethanol bio-refinery. The whole stillage had about 13.1% solids and the wet cake stream was diluted to 13.1% solid with reverse osmosis water. The defatted emulsion samples were added at 0% (control), 0.5%, or 5% levels, incubated at 30.6° C. or 60° C. for 24 hours. Results are shown in FIGS. 6A/B and 7A/B for the wet cake and whole stillage, respectfully. This experiment also evaluated incubation temperatures during the recycle. As shown in the figures for the wet cake recycle, incubation at 60° C. generally did not result in increased liquid phase fat, but an increase of up to 211% in liquid phase fat (at 5% E5) was observed at 30.6° C. incubation (or 141% when using corrected values that subtracts the defatted emulsion fat from the liquid phase). With the whole stillage, incubation at 60° C. also did not result in increased liquid phase fat, but samples E3 & E4 showed enhanced liquid phase fat release and up to 163% increase in liquid phase fat (at 5% E3) at 30.6° C. (or 149% when using corrected values or subtracting defatted emulsion fat from the liquid phase).

### Example 5

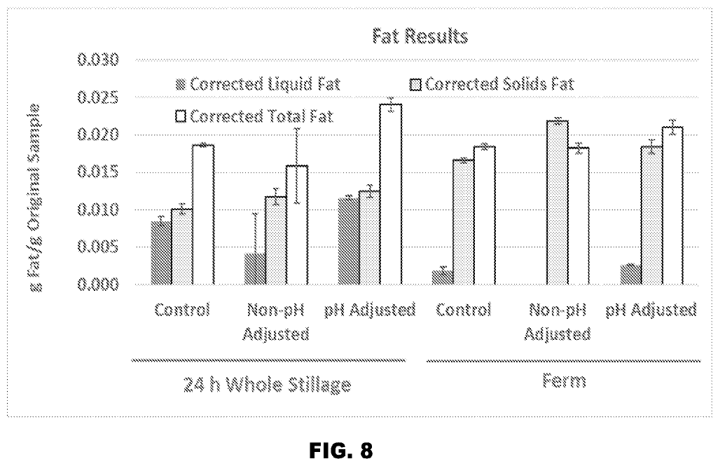

Centrifuge pellets obtained from either pH adjusted defatted emulsion or a non-pH adjusted emulsion were recycled to either a whole stillage stream or to an ethanol fermentation. For whole stillage recycle of the pellets, about 5% of the pellet is incubated with the whole stillage at 30° C. and 250 rpm for about 24 hours. For fermentation recycle of the pellets, about 5% of the pellet is combined with the fermenter. Oil recovery results are shown in FIG. 8.

The present disclosure is not to be limited in scope by the specific embodiments described herein. Indeed, various modifications provided herein in addition to those described herein will become apparent to those skilled in the art from the foregoing description and the accompanying figures. Such modifications are intended to fall within the scope of the appended claims.

It is noted that, as used in this specification and the appended claims, the singular forms “a,” “an,” and “the,” include plural referents unless expressly and unequivocally limited to one referent. Thus, for example, reference to “a component” includes two or more different components. As used herein, the term “include” and its grammatical variants are intended to be non-limiting, such that recitation of items in a list is not to the exclusion of other like items that can be substituted or added to the listed items

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

It is to be understood that each component, compound, substituent or parameter disclosed herein is to be interpreted as being disclosed for use alone or in combination with one or more of each and every other component, compound, substituent or parameter disclosed herein.

It is further understood that each range disclosed herein is to be interpreted as a disclosure of each specific value within the disclosed range that has the same number of significant digits. Thus, for example, a range from 1 to 4 is to be interpreted as an express disclosure of the values 1, 2, 3 and 4 as well as any range of such values.

It is further understood that each lower limit of each range disclosed herein is to be interpreted as disclosed in combination with each upper limit of each range and each specific value within each range disclosed herein for the same component, compounds, substituent or parameter. Thus, this disclosure to be interpreted as a disclosure of all ranges derived by combining each lower limit of each range with each upper limit of each range or with each specific value within each range, or by combining each upper limit of each range with each specific value within each range. That is, it is also further understood that any range between the endpoint values within the broad range is also discussed herein. Thus, a range from 1 to 4 also means a range from 1 to 3, 1 to 2, 2 to 4, 2 to 3, and so forth.

Furthermore, specific amounts/values of a component, compound, substituent or parameter disclosed in the description or an example is to be interpreted as a disclosure of either a lower or an upper limit of a range and thus can be combined with any other lower or upper limit of a range or specific amount/value for the same component, compound, substituent or parameter disclosed elsewhere in the application to form a range for that component, compound, substituent or parameter.

While particular embodiments have been described, alternatives, modifications, variations, improvements, and substantial equivalents that are or can be presently unforeseen can arise to applicants or others skilled in the art. Accordingly, the appended claims as filed and as they can be amended are intended to embrace all such alternatives, modifications variations, improvements, and substantial equivalents.

1. A method for enhancing the corn oil yield in a bio-refinery fermentation process comprising
obtaining an alcohol and by-products including vegetable oil from a grain feedstock through a processing that includes saccharification, fermentation, distillation, and separation to produce the alcohol and the vegetable oil;
isolating an emulsifier in the form of emulsions, defatted emulsions, emulsion precipitates, defatted emulsion precipitates, dried products thereof, or combinations thereof from the by-products; and
recycling at least a portion of the emulsifier to one or more of the saccharification, the fermentation, the distillation, and/or the separation to enhance recovery of the vegetable oil.

2. The method of claim 1, wherein the emulsifier includes at least a protein of at least one of globulin-1 S allele precursor, globulin-1, globulin 2 precursor, globulin-1 S allele-like, oleosin Zm-I, oleosin Zm-II, zeamatin-like protein, granule-bound starch synthase, trypsin inhibitor, and gamma zein (16 kDa), and mixtures thereof.

3. The method of claim 1, wherein the emulsifier is an oil emulsion and includes at least a protein of Globulin-1 S allele precursor, Globulin-1, Globulin 2, Oleosin Zm-I, Oleosin Zm-II, Zeamatin-like protein, Granule-bound starch synthase, Trypsin inhibitor, gamma zein, or mixtures thereof.

4. The method of claim 1, wherein the emulsifier is a defatted emulsion precipitate and includes at least a protein of Globulin-1 S allele precursor, Globulin-1, Globulin 2, Globulin-1 S allele-like, Oleosin Zm-I, Oleosin Zm-II, Trypsin inhibitor, Zeamatin-like protein, Granule-bound starch synthase, or combinations thereof.

5. The method of claim 1, wherein the emulsifier further includes at least one of minerals, modified lipids produced during fermentation, and/or non-germ protein.

6. The method of claim 1, wherein the emulsifier is a by-product of a bio-refinery process obtained from a bio-refinery process stream selected from the group consisting of beer, whole stillage, thin stillage, and syrup.

7. The method of claim 1, wherein the saccharification and fermentation occur simultaneously.

8. The method of claim 1, wherein the recycling is introduced into one or more of a slurry stream, a fermentation vessel, a beer well, and a stillage stream.

9. The method of claim 1, wherein the emulsifier includes (a) less than about 10% protein, preferably about 1% to about 5% protein; (b) about 30% to about 60% grain oil; and (c) about 40% to about 60% water.

10. The method of claim 1, wherein the emulsifier is dried and includes (a) about 2 to about 15% protein; (b) about 60% to about 85% grain oil; and (c) less than about 10% water, preferably about 0.5% to about 10% water.

11. The method of claim 1, wherein the emulsifier is defatted and includes (a) about 2 to about 15% protein; (b) about 5% to about 20% grain oil; and (c) about 60% to about 90% water.

12. The method of claim 1, wherein the emulsifier is dried and defatted and includes (a) about 10 to about 60% protein, preferably about 10 to about 40% protein, or preferably about 30 to about 60% protein; (b) about 20% to about 45% grain oil, preferably about 20% to about 40% grain oil; and (c) less than about 15% water, preferably about 0.5% to about 15% water.

13. The method of claim 1, wherein the emulsifier is a precipitate or defatted precipitate and includes and includes (a) about 30 to about 60% protein; (b) about 5% to about 20% grain oil; and (c) less than about 15% water, preferably about 0.5% to about 15% water.

14. An emulsion for enhancing the corn oil yield in a bio-refinery fermentation process, the emulsion comprising
a process stream from a bio-refinery fermentation and including at least one of a saccharification stream, a fermentation stream, a beer stream, a whole stillage stream, a wet cake stream, a thin stillage stream, a syrup stream, or combinations thereof;
an emulsifier in the form of emulsions, defatted emulsions, emulsion precipitates, defatted emulsion precipitates, dried products thereof, or combinations thereof isolated from by-products of the bio-refinery fermentation.

15. The emulsion of claim 14, wherein the emulsifier includes at least a protein of at least one of globulin-1 S allele precursor, globulin-1, globulin 2 precursor, globulin-1 S allele-like, oleosin Zm-I, oleosin Zm-II, zeamatin-like protein, granule-bound starch synthase, trypsin inhibitor, and gamma zein (16 kDa), and mixtures thereof.

16-21. (canceled)

22. The emulsion of claim 14, wherein the emulsifier includes (a) less than about 10% protein, preferably about 1% to about 5% protein; (b) about 30% to about 60% grain oil; and (c) about 40% to about 60% water.

23. The emulsion of claim 14, wherein the emulsifier is dried and includes (a) about 2 to about 15% protein; (b) about 60% to about 85% grain oil; and (c) less than about 10% water, preferably about 0.5% to about 10% water.

24. The emulsion of claim 14, wherein the emulsifier is defatted and includes (a) about 2 to about 15% protein; (b) about 5% to about 20% grain oil; and (c) about 60% to about 90% water.

25. The emulsion of claim 14, wherein the emulsifier is dried and defatted and includes (a) about 10 to about 60% protein, preferably about 10 to about 40% protein, or preferably about 30 to about 60% protein; (b) about 20% to about 45% grain oil, preferably about 20% to about 40% grain oil; and (c) less than about 15% water, preferably about 0.5% to about 15% water.

26. The emulsion of claim 14, wherein the emulsifier is a precipitate or defatted precipitate and includes (a) about 30 to about 60% protein; (b) about 5% to about 20% grain oil; and (c) less than about 15% water, preferably about 0.5% to about 15% water.
